(19) United States
(12) Patent Application Publication
(10) Pub. No.: US 2018/0099471 A1
(43) Pub. Date: Apr. 12, 2018

(54) MACHINE FOR PRODUCING A CORRUGATED-BOARD WEB

(71) Applicant: BHS Corrugated Maschinen- und Anlagenbau GmbH, Weiherhammer (DE)

(72) Inventors: Klaus BAIERL, Schnaittenbach (DE); Norbert STÄDELE, Parkstein (DE)

(21) Appl. No.: 15/566,404

(22) PCT Filed: Apr. 11, 2016
(86) PCT No.: PCT/EP2016/057928

§ 371 Date: Oct. 13, 2017

(30) Foreign Application Priority Data
Apr. 14, 2015 (DE) 10 2015 206 650.9

(51) Int. Cl. B31F 1/28; G01N 22/02

(52) U.S. Cl. CPC B31F 1/2818; G01N 33/346; G01N 22/02; B31F 1/2831

(57) ABSTRACT

A machine for producing a corrugated-board web with at least one cover web and at least one corrugated web connected with the same by a gluing. The machine includes at least one microwave quality determining unit for determining at least one dielectric characteristic of the corrugated-board web for determining the quality of the same by microwaves.

### CROSS REFERENCE TO RELATED APPLICATIONS

This application is a United States National Phase application of International Application PCT/EP2016/057928 filed Apr. 11, 2016 and claims the benefit of priority under 35 U.S.C. § 119 of German patent application 10 2015 206 650.9 filed Apr. 14, 2015, the entire contents of which are incorporated herein by reference.

### FIELD OF THE INVENTION

The invention concerns a machine for producing a corrugated-board web with several individual webs. The invention further concerns a quality determining means as a component of such a machine.

### BACKGROUND OF THE INVENTION

Machines with different quality determining means are already known from prior art for checking the quality of a produced corrugated-board strip. Vibration or ultrasound sensors can for example be used for this.

### SUMMARY OF THE INVENTION

The invention is based on the task of creating a corrugated-board machine in which the quality of a corrugated-board web is determinable as easily and as simply as possible. A corresponding quality determining means as a component of such a corrugated-board machine is also to be provided.

This task is solved according to the invention by a machine for producing a corrugated-board web with at least one cover web and at least one corrugated web connected to the same by means of at least one gluing, comprising at least one microwave quality determining means for determining at least one dielectric characteristic of the corrugated-board web for determining its quality by means of microwaves, and by a microwave quality determining unit as a component of a machine according to the invention, designed in such a way that at least one dielectric characteristic of the corrugated-board web is determinable. The core of the invention consists of the at least one quality determining means using microwaves for determining the quality of the corrugated-board web.

Microwaves are known to be electromagnetic waves. Thanks to their wavelengths, microwaves are particularly suitable for exciting a dipolar and multipolar vibration of molecules, such as water molecules. The measurement is therefore physically substantially based on the evaluation of the dipolar relaxation of the water molecules in the corrugated-board web. Water molecules align themselves in an outwardly located field in a preferred direction and can therefore also be polarized. If an electromagnetic alternating field is applied the water molecules in the corrugated-board web will begin to rotate with the frequency of the alternating field. This effect is comprising the dielectric constant.

The at least one microwave quality determining means determines at least one dielectric characteristic of the corrugated-board web, in particular its humidity and/or density, for determining the quality of the corrugated-board web. Depending on the determined at least one dielectric characteristic of the corrugated-board web, its quality is determinable. The quality of the corrugated-board web is for example dependent on the humidity of the corrugated-board web. The quality of the corrugated-board web can in particular be determined across its entire width.

It can thus for example be determined whether a fault exists in at least one gluing and/or in at least one individual web of the corrugated-board web. In particular it is determinable whether the at least one gluing is faulty in its application quantity and/or distribution. It can for example be determined in this way whether the corrugated-board web comprises at least one undesired hollow space in at least in some areas and/or at least one undesired additional material layer in some areas. It can for example be determined in this way whether the corrugated-board web comprises at least one incorrect material layer in at least some areas. It can also conveniently be determined whether incorrect streams are present at least in some areas. It is of advantage if the maximum production or transport speed of the corrugated-board web is taken into consideration or determined during an evaluation.

It is of advantage if a machine for producing a corrugated-board web laminated on one side is accelerated until the at least one microwave quality determining means determines a problem or problems with the gluing of individual webs of the corrugated-board web laminated on one side. The speed of the machine for producing the corrugated-board web is then preferably reduced again until the at least one microwave quality determining means no longer detects such a problem or no such problems anymore. The maximum production speed of the corrugated-board web is thus available or can be realized for a desired quality of the corrugated-board web.

It is of advantage if the at least one microwave quality determining means draws a conclusion regarding the reason for the fault from recurring faults and reduces or rectifies the reason for the fault accordingly.

The at least one microwave quality determining means preferably uses the reflection, irradiation and/or resonance method. Resonant or non-resonant methods, transmission or reflection methods and/or scatter field or irradiation field methods can thus for example be used.

If at least one microwave resonator is present, its frequency will change when a fault exists in the corrugated-board web. The resonance frequency in particular will fall as humidity increases. Resonant measuring methods utilize the resonance parameters of a construct capable of vibrating, in general microwave resonators.

It is of advantage if the microwaves penetrate the entire corrugated-board web across its thickness when using the transmission method. The corrugated-board web is irradiated with the transmission method, so that one can determine an integral humidity value across the volume of the corrugated-board web, and therefore easily across lower homogeneities. Microwave parameters that can be used for transmission measurement are the damping and/or phase offset of the electromagnetic wave running through the corrugated-board web.

The electromagnetic field of a scatter field arrangement preferably extends into the corrugated-board web. Measured is the electromagnetic wave extending into the corrugated-board web as well as the wave reflected by the corrugated-board web. In principle, access to the corrugated-board web is required from one side of said corrugated-board web only.

The microwaves preferably run vertical to a transport direction of the corrugated-board web. They favorably run substantially vertical to its surface.

The microwave quality determining means works in a contactless way.

It is of advantage if the at least one microwave quality determining means is/are displaceable in their entirety, or individual components of the same.

The at least one microwave quality determining means favorably determines the at least one dielectric characteristic of the corrugated-board web during the transport of said corrugated-board web.

The signal evaluation unit is for example a component of the microwave sensor. Alternatively it can also be separate from the microwave sensor.

The at least one microwave quality determining means is favorably arranged between the machine for producing the corrugated-board web laminated on one side and a pre-heating means. Alternatively and/or in addition, at least one microwave quality determining means is arranged between the heated pressure means and a cross-cutter means.

The corrugated-board web favorably consists of at least two twin-layer, preferably three-layer, five-layer or seven-layer corrugated-board web.

It is of advantage if the at least one cover web is substantially smooth, whilst the at least one corrugated-board is corrugated.

The design in which the at least one microwave quality determining means is arranged in such a way that it determines at least one dielectric characteristic of the corrugated-board web at least in the area of its at least one gluing allows a particularly comprehensive determination of the quality of the corrugated-board web. In particular, the quality of the at least one gluing is determinable especially well and easily.

According to one embodiment the at least one microwave quality determining means has at least one microwave sensor. It is of advantage if the at least one microwave sensor is equipped with at least one reflector for reflecting transmitted and/or reflected microwaves. A design without reflector is a possible alternative.

The at least one microwave sensor is preferably arranged on a support. It is expedient if several supports with microwave sensors are arranged behind each other in the transport direction of the corrugated-board web.

According to one embodiment the at least one microwave sensor has at least one microwave transmitter or microwave generator for generating at least one microwave field for interacting with the corrugated-board web.

According to one embodiment the at least one microwave sensor comprises at least one receiver unit for receiving the microwaves of the at least one microwave transmitter. The at least one microwave transmitter and the at least one receiver unit allocated to the same for receiving its microwaves are for example arranged opposite each other or on different sides of the corrugated-board web, so that the corrugated-board web runs between the same.

Alternatively the at least one receiver unit and the at least one microwave transmitter are arranged next to each other. They are arranged on a common side of the corrugated-board web or face a common side of the corrugated-board web. At least one reflector is then preferably present for reflecting the microwaves generated by the at least one microwave transmitter through the corrugated-board web back to the at least one receiver unit.

It is of advantage if several microwave sensors are provided. These are then favorably arranged next to each other across a transverse direction of the corrugated-board web, so that the quality of the corrugated-board web can preferably be determined across the entire width of the same. Several microwave transmitters and receiver units are therefore favorably envisaged next to each other. The distance of the microwave transmitters from each other is preferably constant.

The at least one signal evaluation unit for signal evaluation is favorably capable of converting a signal from the microwave receiver unit or the microwave sensor into a humidity value of the corrugated-board web. It is favorably in signal connection with the microwave receiver unit or the microwave sensor.

By determining local changes in the at least one dielectric characteristic of the corrugated-board web faults in the corrugated-board web can be recognized extremely quickly and precisely. Floating averaging is preferably omitted.

The determining of the at least one hollow space with the at least one microwave quality determining means is favorably not dependent on a measured humidity of the corrugated-board web and/or the quality of the gluing of the corrugated-board web.

The at least one microwave quality determining means according to one embodiment is favorably self-learning, in other words it deduces a reason of a fault from recurring faults and reduces or rectifies the reason for the fault.

The at least one microwave sensor that includes the at least one signal evaluation unit is preferably designed as a combination unit.

The design that comprises at least one support for enveloping a relevant edge of the corrugated-board web, wherein the at least one support comprises a first leg, on which at least one microwave transmitter is arranged, comprises a second leg, on which at least one associated receiver unit is arranged, and comprises an incline around an angle that substantially equals a flank angle of a corrugated web of the corrugated-board web, so that the microwaves are substantially transmitted through a flank of the corrugated web, and independently sets itself to the relevant flank angle of the corrugated web allows an automatic, independent or in particular fast adjustment of the at least one microwave quality determining unit or the at least one microwave sensor to a respective flank angle of the corrugated web.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a first part of a corrugated-board machine according to a first embodiment example;

FIG. 2 is a schematic view of a second part of the corrugated-board machine according to a first embodiment example;

FIG. 3 is a view showing an overview of a microwave quality determining means in the corrugated-board machine according to FIGS. 1 and 2;

FIG. 4 is a sectional view along section line IV-IV in FIG. 3;

FIG. 5 is a sectional view along section line V-V in FIG. 4;

FIG. 6 is a view of a stress/time diagram produced by a microwave receiver unit of the microwave quality determining means according to FIG. 3 in the area of a faulty gluing;

FIG. 7 is a view of a second embodiment example according to the invention equaling that of FIG. 3;

FIG. 8 is a view of a third embodiment example according to the invention equaling that of FIG. 3;

FIG. 9 is a view of a fourth embodiment example according to the invention equaling that of FIG. 5; and

FIG. 10 is a view of a fifth embodiment example according to the invention equaling that of FIG. 5.

### DESCRIPTION OF THE PREFERRED EMBODIMENTS

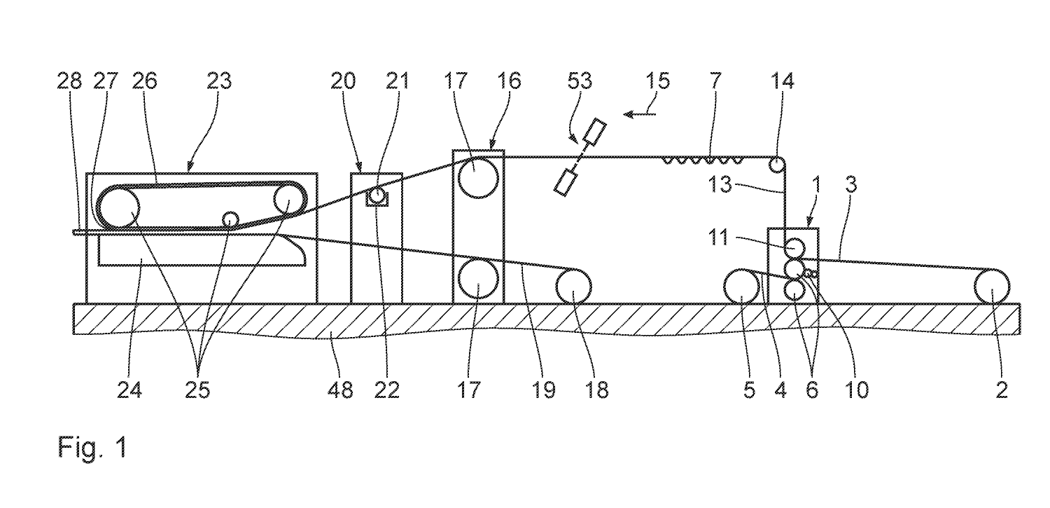

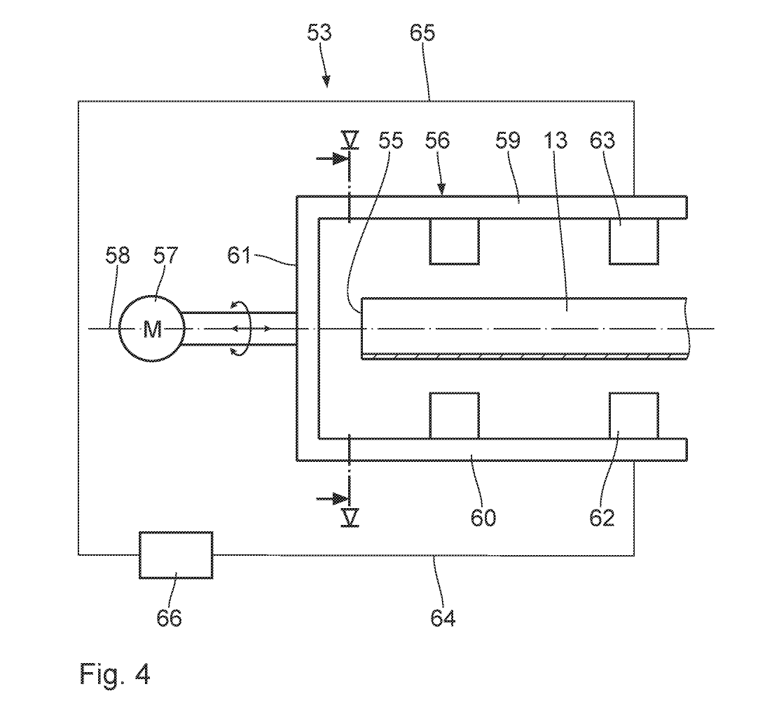

A first embodiment example will be described hereafter with reference to FIGS. 1 to 6. A corrugated-board machine like the one schematically illustrated in FIG. 1, 2 comprises a machine 1 for producing a corrugated-board web laminated on one side.

A first, preferably endless material web 3 is supplied to the machine 1 from a first unwinding unit 2. The material web 3 represents a first cover web for the corrugated-board web produced in the machine 1.

The first material web 3 is joined to a second, preferably endless material web 4 in the machine 1, which is unwound from a second unwinding unit 5. After unwinding, the second material web 4 is routed between two corrugated rolls 6 arranged next to each other for creating corrugation in the machine 1. Following this process the second material web 4 is present as a corrugated web 7. The latter alternately comprises corrugation peaks 8 and corrugation troughs 9.

Glue is then applied to the corrugated web 7 in a gluing unit 10 of the machine 1. It is compressed with the first material web 3 in a gap between the pressing roller 11 and one of the corrugated rolls 6 in the machine 1, which joins them together and creates a gluing 12. The corrugated-board web 13 laminated on one side that is created from the cover web 3 and the corrugated web 7 is extracted at the top and diverted around a deflection roller 14 in a working direction 15. The machine 1 for producing the corrugated-board web 13 laminated on one side is generally known, for example from EP 0 687 552 A2 (equals U.S. Pat. No. 5,632,850, the entire contents of which are incorporated herein by reference), from DE 195 36 007 A1 (equals GB 2,305,675 A, the entire contents of which are incorporated herein by reference) or from DE 43 05 158 A1, to which we refer for details and the entire contents of each of these references are incorporated herein by reference.

A pre-heating means 16 is arranged downstream from the machine 1 in working direction 15. This comprises two heatable heating rollers 17 arranged one above the other.

A second unwinding unit 18 for a third, preferably endless material web 19 is arranged before the pre-heating means 16, from which the same is unwound and transported in the working direction 15 by the pre-heating means 16. The one-sided corrugated-board web 13 and the third material web 19 both partially envelope the heating rollers 17 and are guided in the working direction 15 past the same.

A gluing unit 20 with a gluing roller 21 is arranged behind the pre-heating means 16 in working direction 15 and is partly submersed in a glue bath 22. The corrugated web 7 of the corrugated-board web 13 is in contact with the gluing roller 21 for applying glue.

A heated pressure means 23 is arranged behind the gluing unit 20 and comprises a horizontal table 24 extending in working direction 15 with heating plates (not shown). A driven endless pressure belt 26 guided over three rollers 25 is envisaged above the table 24. A pressure gap 27 is formed between the pressure belt 26 and the table 24, through which the corrugated-board web 13 and the third material web 19 are guided and pressed against each other. A corresponding heated pressure means 23 is known from DE 199 54 754 Al, the entire contents of which are incorporated herein by reference. A three-layer corrugated-board web 28 is formed in the heated pressure means 23.

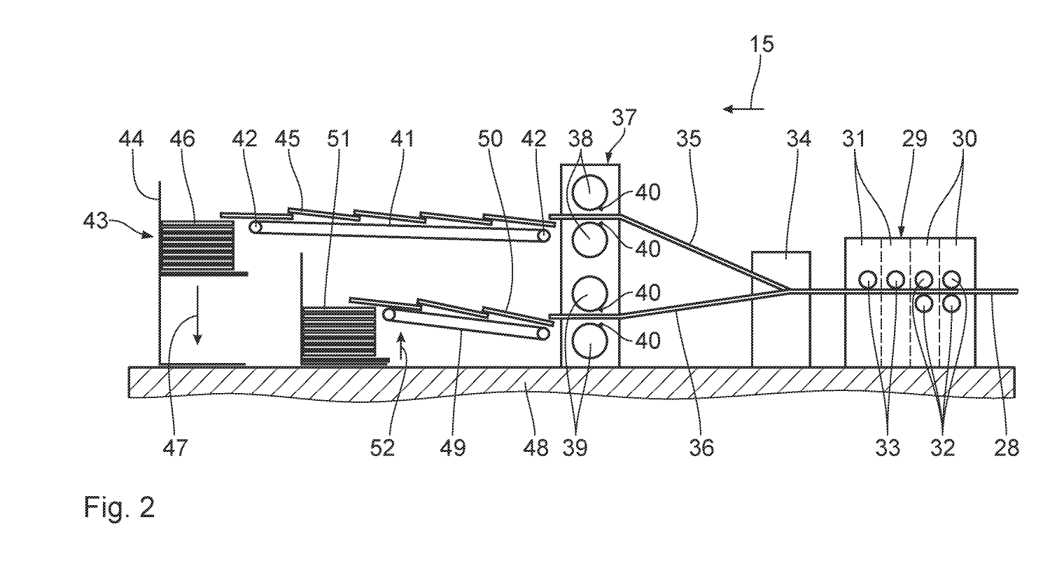

FIG. 2 shows a second part of the corrugated-board machine following the exit of the corrugated-board web 28 from the heated pressure means 23. There follows a longitudinal cutting and grooving means 29, which comprises two grooving stations 30, one behind the other, and two longitudinal cutting stations 31, one behind the other. The grooving stations 30 each comprise pairs of grooving tools 32 arranged one above the other, between which the corrugated-board web 28 is passed through. The longitudinal cutting stations 31 each have rotary driven blades 33 that can be engaged with the corrugated-board web 28 for longitudinally separating the same. The detailed construction of the longitudinal cutting and grooving means 29 is known from DE 197 54 799 A1 (equals U.S. Pat. No. 6,071,222, the entire contents of which are incorporated herein by reference) and from DE 101 31 833 A1, to which we herewith refer with regard to construction details and the entire contents of each reference are incorporated herein by reference.

A switch 34 in which longitudinally cut web sections 35, 36 of the corrugated-board web 28 are separated from each other is arranged behind the longitudinal cutting and grooving means 29 in working direction 15.

The web sections 35, 36 are then forwarded to a transverse cutting means 37. This comprises an upper transverse cutting roller pair 38 for the upper web section 35 and a lower transverse cutting roller pair 39 for the lower web section 36. The rollers of the roller pairs 38, 39 each support a radially outwardly extending blade 40 that runs vertical to the working direction 15. The blades 40 of a transverse cutting roller pair 38, 39 act together for separating the web sections 35, 36.

The upper transverse cutting roller pair 38 is followed by an upper conveyor belt 41, which is guided around rotatably driven rollers 42.

A shelf 43 with a vertically extending shoulder 44 is arranged behind the upper conveyor belt 41, on which corrugated-board sheets 45 cut from the web section 35 by means of the transverse cutting means 37 are stacked to form a stack 46. As indicated by the directional arrow 47 the shelf 43 is height adjustable. The shelf 43 can in particular be lowered to a machine floor 48 that supports the corrugated-board machine for the onward transport of the stack 46.

The lower transverse cutting roller pair 39 is followed by a further, lower conveyor belt 49 that stacks corrugated-board sheets 50 cut from the web section 36 by means of the transverse cutting means 37 on a further shelf 51. The lower conveyor belt 49 can be lifted for adjusting the height of the stack, as is indicated by a directional arrow 52.

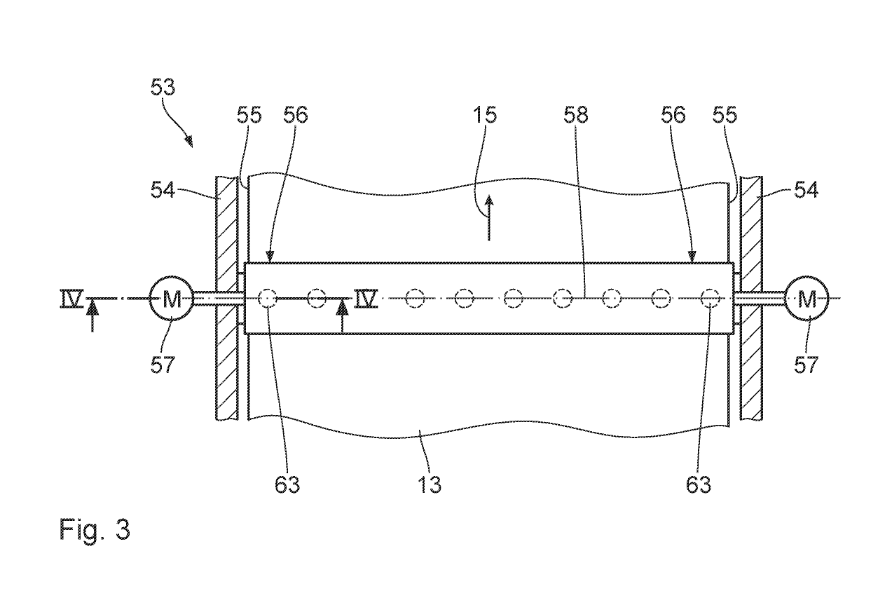

The corrugated-board machine also comprises a microwave quality determining means 53 illustrated in more detail in FIGS. 3 to 5. This serves amongst other things for determining the quality of the gluing 12 between the corrugated-board web 7 and the cover web 3. The means 53 is here arranged behind the machine 1 and before the heated pressure means 23, in which the third material web 19 is pressed on, in working direction 15. Preferred is a placement between the machine 1 and the pre-heating means 16.

The means 53 comprises braced supports 54 on the machine floor 48 on both sides of the corrugated-board web 13. A support 56, favorably with a U-shaped cross-section, is arranged at both supports 54 and preferably envelopes the relevant edge 55 of the corrugated-board web 13 and is braced against the supports 54. Outside of the supports 54 a drive 57 each is preferably envisaged, which favourably enables a pivoting around a pivot axis 58. The pivot axis 58 then lies centrally in the corrugated-board web 13 and extends vertical to the working direction 15. Each drive 57 preferably further allows a displacement of the neighboring support 56 along the pivot axis 58, so that the same support 56 can be pushed around the edge 55 of even smaller widths of the corrugated-board web 13. Each support 56 comprises two legs 59, 60 extending parallel to each other, which are preferably connected with each other by a common base plate 61 extending vertically to the same, and is designed as a single part with the same. It is preferred that the two supports 56 are permanently connected with each other via their legs 59, 60, so that the supports 56 form a joined, solid support arrangement. The support arrangement extends along the transverse direction of the corrugated-board web 13.

Several microwave transmitters 62 are arranged on the inside of the leg 60. Associated microwave receiver units 63 are arranged on the opposite inside of the relevant leg 59. Each transmitter 62 and the associated receiver unit 63 are in the same transversal position in relation to the corrugated-board web 13, i.e. they have the same vertical distance from the edge 55 of the corrugated-board web 13. Each transmitter 62 is connected via a line 64, and each receiver unit 63 via a line 65 with a common signal evaluation unit 66 in a data transmitting way.

Each transmitter 62 and the associated receiver unit 63 lie on a common central longitudinal straight 67. The second material web 4 lies on a plane 68 defining the same. The straight 67 encloses an angle b with the plane 68. The angle b can be set, where envisaged, by pivoting the relevant support 56 around the axis 58.

A flank 69 of the corrugated-board web 13, which encloses an angle c with the plane 68, is located between each wave peak 8 and a wave trough 9. The angle b is selected in such a way that it equals the angle c as far as possible. This means that the microwaves are transmitted via the flank 69 of the corrugated-board web 7 itself as much as possible, and not via the surrounding air. In principle the following applies for angle b: 0°≦b≦90°, in particular 0°<b<90°, in particular 15°≦b≦65°, in particular 35°≦b≦45°, in particular b≈40°. By pivoting the relevant support 56 the angle b can be adapted to the flank angle c for various corrugated board types. Setting the relevant support 56 to flank angle c is preferably realized independently or automatically. At least one corresponding flank angle determining sensor is favorably provided for this.

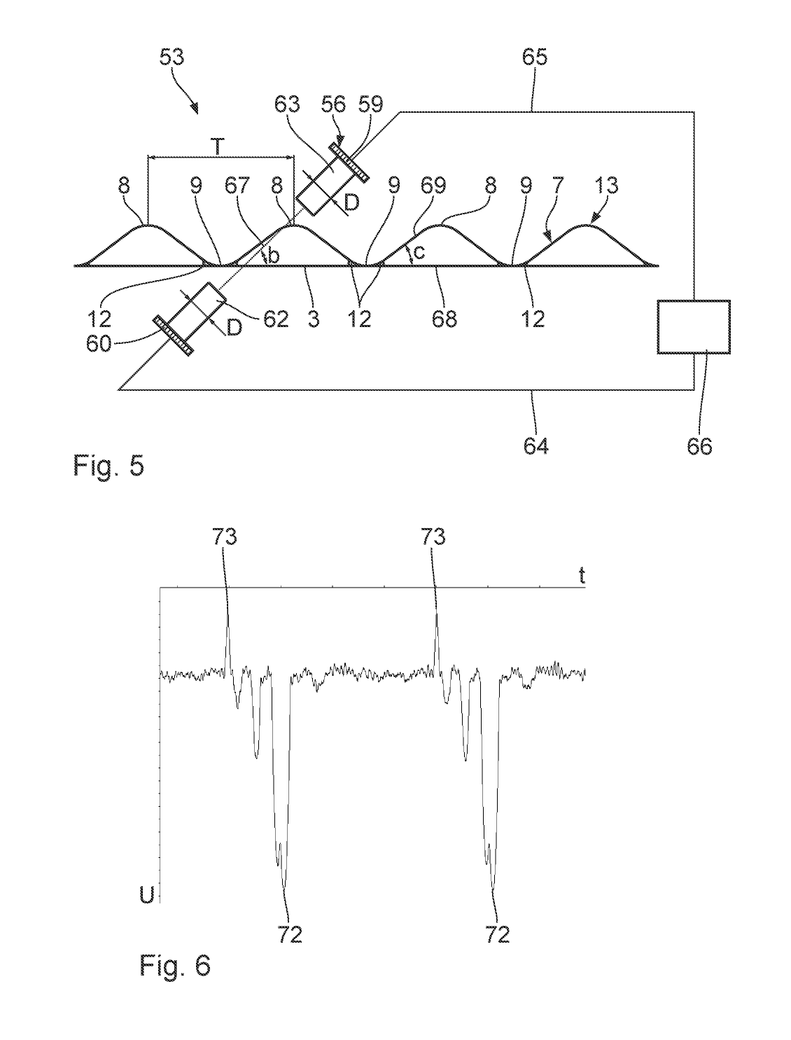

Every microwave transmitter 62 for example works at a frequency of between 300 MHz and 300 GHz and transmits a corresponding microwave field during its operation. The illustration in FIG. 5 is not to scale in that the corrugated-board web 13 is illustrated in an overproportional way. In principle the corrugated-board web 13 comprises a division T. The transmitter 62 and receiver units 63 each have a diameter D. The diameter D is preferably larger than the division T. Typical diameters D are 20 mm to 60 mm.

The positions of the transmitters 62 or receiver units 63 can also be interchanged. In addition it is possible to envisage an arrangement that results if one rotates the transmitter 62 and receiver units 63 by an angle of 180°-2b around the intersection between the straight 67 and the plane 68 in an anti-clockwise direction. This interchanges and reverses the upstream position of the transmitter 62 or receiver units 63 to a corresponding downstream position. The proportionate angle b does however remain, and is now measured on the left of the straight 67 and not on the right of the same, as in FIG. 5.

The functionality of the microwave quality determining means 53 will now be described hereafter with reference to FIG. 6. As soon as the machine 1 has produced the corrugated-board web 13 laminated on one side, the same is guided through the means 53 at a previously known speed. The transversal positions of the supports 56 are both set in such a way for this that they envelope the two edges 55 of the corrugated-board web 13, but to not touch it.

The microwave transmitters 62 constantly transmit microwaves, which are received by the associated microwave receiver unit 63. Microwave fields are generated in this way. The corrugated-board web 13 runs through the microwave fields. Due to the fact that microwaves are transmitted much better through mass, i.e. through paper or cardboard, than through air a pulsating signal is generated in the case of a perfect gluing 12 depending on whether the signal, as in the case of FIG. 5, is currently being transmitted mostly through the flank 69 or needs to be transmitted mainly through air. In angle b=90° the differences in signal intensity at the relevant receiver unit 63 between a wave peak 8 that has just passed through and a wave trough 9 will be very small, as the microwaves would have to be mainly transmitted through air in both cases, either between the material web 3 and the wave peak 8 or above the wave trough 9. Signal evaluation would be extremely complicated in this case. The incline of the relevant support 56 by an angle of b<90°, which substantially equals the flank angle c, ensures that the microwaves are transmitted mainly through the flank 69, the gluing 12 and the material web 3 when they pass the wave peak 8, so that a particularly large signal is generated with a correct gluing 12, which will drop accordingly when the corrugated-board web 13 is transported onwards.

If a faulty gluing 12 exists or if a so-called flank break has occurred, a signal of a much lower intensity will be present at the point where a larger signal would have been expected. This signal can be simply converted into a digital signal by stipulating certain threshold values, wherein “1” means production fault and “0” no fault. FIG. 6 shows the strong voltage drop when a faulty gluing 12 passes through, with breakouts 72 in a downward direction. FIG. 6 also shows the strong voltage increase when missing layers, webs, layer areas or web areas pass through, with breakouts 73 in an upward direction. This signal can also be easily converted into a digital signal by stipulating certain threshold values, wherein “1” means production fault and “0” no fault.

Deductions regarding the firmness and quality of the corrugated-board web 13 can be made from the values obtained.

It is favorable that the means 53 works contactless and without inertia. No huge electronic effort is required for the simple threshold value analysis of the signals of the relevant receiving unit 63, so that an on-line determining of the gluing quality will be possible even at very high material web speeds, for example 400 m/min.

Areas or sections of the corrugated-board web 13 determined by the microwave quality determining means 53 as deviating from a target condition of the corrugated-board web 13 are for example excluded from the procedure or process or marked. Alternatively these sections or areas remain in the procedure and are not excluded.

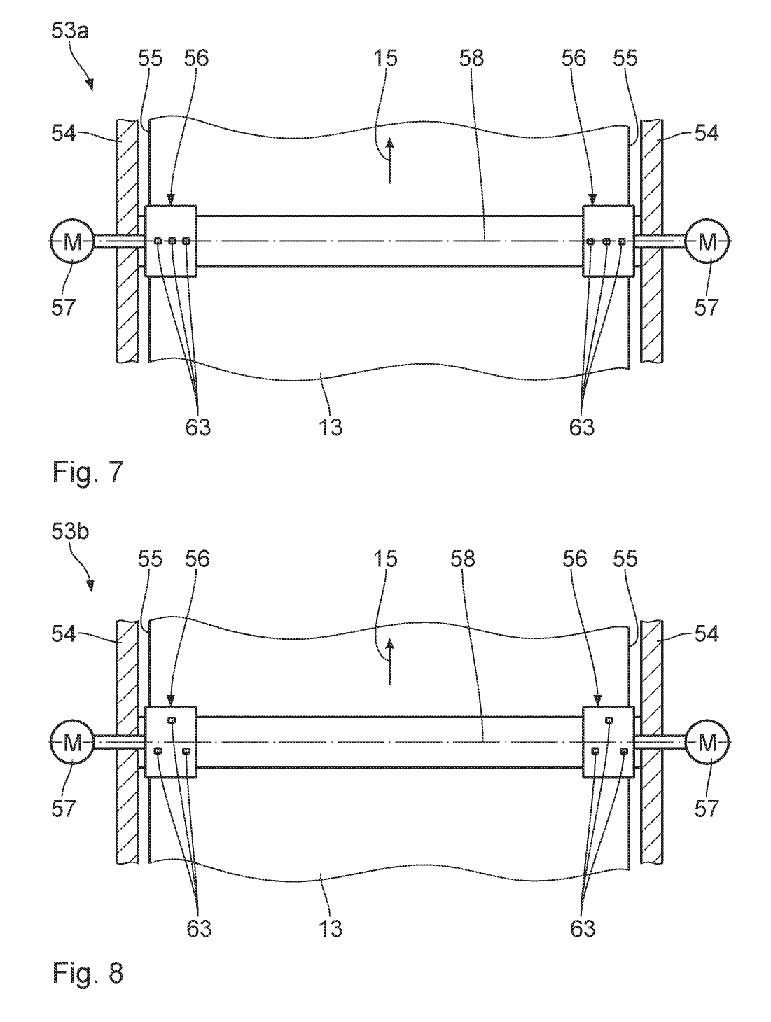

A second embodiment example will be described hereafter with reference to FIG. 7. Identically constructed parts are identified with the same reference numbers than for the first embodiment example, to the description of which we herewith refer. Differently constructed, although functionally identical parts are identified with the same reference numbers with the addition of an a. The microwave quality determining means 53a comprises several transmitters 62 arranged in a line next to each other, and several receiver units 63 correspondingly arranged opposite in a line next to each other. This has the advantage that the quality of the gluing 12 can be examined across a greater width. In principle it is possible to design the legs 59 and 60 so long as both supports 56 together envelope the entire corrugated-board web 13. This applies for all embodiment examples. It is further possible to arrange the transmitters 62 and the associated receiver units 63 transversally displaceable on a trolley, i.e. transverse to the working direction 15. In this way it is also possible to examine the quality of the gluing 12 across the complete width of the corrugated-board web 13.

A third embodiment example will be described hereafter with reference to FIG. 8. Identical parts are identified with the same reference numbers as for the first embodiment example. Differently constructed, although functionally identical parts are identified with the same reference numbers with the addition of a b. The main difference compared to the second embodiment example consists of the microwave quality determining means 53b also comprising several transmitters 62 and several associated receiver units 63, which are however not arranged in a line but in a zigzag arrangement. This has the advantage that more transmitter 62 or receiver units 63 can be arranged on a predetermined length of the legs 59, 60 transverse to the working direction 15, and can also be arranged closer to each other. A minimum distance to the next neighboring transmitter is required for each transmitter 62 with a predetermined diameter D, so that the signals will not overlap on the receiver side. The arrangement according to FIG. 8 allows the positioning of more transmitters 62 on a predetermined leg length, so that a more precise analysis of the gluing quality is also possible.

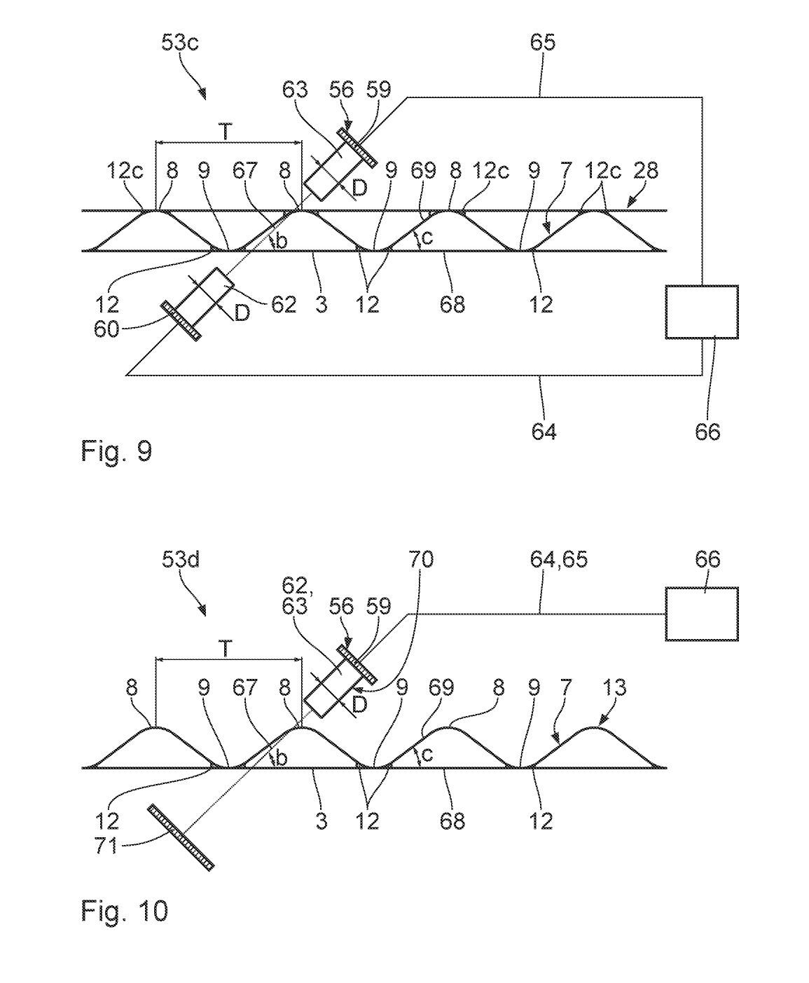

A fourth embodiment example will be described hereafter with reference to FIG. 9. Identically constructed parts are identified with the same reference numbers as for the first embodiment example, to the description of which we herewith refer. Differently constructed, although functionally identical parts are identified with the same reference numbers with the addition of a c. The main difference compared to the first embodiment example consists of the quality determining means 53c serving the microwave quality determining of a corrugated-board web 28 consisting of three material webs or more. Accordingly the means 53c is arranged behind the heated pressure means 23 and before the cross-cutter means 37 in working direction 15. Preferred is an arrangement between the longitudinal cutting and grooving means 29 and the switch 34. The means 53c is substantially constructed like the means 53 according to the first embodiment. The corrugated-board web 28 to be examined merely has more material webs. More gluings 12, 12c therefore need to be checked for quality. In principle the functionality of fault recognition is the same. If gluings 12, 12c are faulty, the transmission of the microwaves is lower there than with a perfect gluing. The means 53c can also be used for examining corrugated-board webs with wider material webs, for example a corrugated-board web with three smooth material webs and two corrugated webs.

A fifth embodiment example will be described hereafter with reference to FIG. 10. Identically constructed parts are identified with the same reference numbers as for the first embodiment example, to the description of which we herewith refer. Differently constructed, although functionally identical parts are identified with the same reference numbers with the addition of a d. The main difference compared to the first embodiment example consists of the quality determining means 53d of the microwave transmitter 62 and the microwave receiver unit 63 being arranged directly next to each other. They are joined to form one unit 70. The microwave transmitter 62 and the microwave receiver unit 63 face a common first side of the corrugated-board web 13. The microwave transmitter 62 and the microwave receiver unit 63 are thus arranged on a common side of the corrugated-board web 13.

A reflector 71 faces the second side of the corrugated-board web 13 that lies opposite the first side of the corrugated-board web 13. The corrugated-board web 13 thus runs between the unit 70 including the microwave transmitter 62 and the microwave receiver unit 63 and the reflector 71. The microwave transmitter 62 once again transmits constant microwaves during operation, which penetrate the corrugated-board web 13 and hit the reflector 71. The reflector 71 reflects the microwaves back, so that the microwaves once again permeate the corrugated-board web 13 and are received by the microwave receiver unit 63. The corrugated-board web 13 is thus quasi permeated twice by the microwaves.

A design with a reflector 71 is alternatively also possible with the preceding embodiment examples, in particular with the design according to FIG. 9.

While specific embodiments of the invention have been shown and described in detail to illustrate the application of the principles of the invention, it will be understood that the invention may be embodied otherwise without departing from such principles.

1. A machine for producing a corrugated-board web with at least one cover web and at least one corrugated web connected to the at least one cover by means of at least one gluing the machine comprising:
at least one microwave quality determining means for determining at least one dielectric characteristic of the corrugated-board web for determining a quality of the corrugated-board web by microwaves.

2. A machine according to claim 1, wherein the at least one microwave quality determining means works with the microwaves, wherein a wavelength of the microwaves lies between 1 m and 1 mm.

3. A machine according to claim 1, wherein the at least one microwave quality determining means is arranged such that the at least one microwave quality determining means determines at least one dielectric characteristic of the corrugated-board web at least in an area of at least one gluing of the corrugated-board web.

4. A machine according to claim 1, wherein the at least one microwave quality determining means comprises at least one microwave sensor.

5. A machine according to claim 1, wherein the at least one microwave quality determining means comprises at least one signal evaluation unit for signal evaluation.

6. A machine according to claim 1, wherein local changes in the quality of the corrugated-board web are detectable with the at least one microwave quality determining means.

7. A machine according to claim 1, wherein the at least one microwave quality determining means determines whether the corrugated-board web comprises at least one undesired hollow space in at least one area.

8. A machine according to claim 1, wherein the at least one microwave quality determining means determines whether the corrugated-board web comprises an undesired additional material layer in at least one area.

9. A machine according to claim 1, wherein the at least one microwave quality determining means determines whether the corrugated-board web comprises at least one incorrect material layer in at least one area.

10. A machine according to claim 1, wherein the at least one microwave quality determining means determines whether incorrect floods are present in at least one area.

11. A machine according to claim 1, wherein the at least one microwave quality determining means deduces a reason of a fault from recurring faults and reduces or rectifies the reason for the fault.

12. A machine according to claim 5, wherein the at least one signal evaluation unit is part of at least one microwave sensor.

13. A machine according to claim 1, wherein areas or sections of the corrugated-board web determined by the at least one microwave quality determining means as deviating from a target condition of the corrugated-board web are excluded from a procedure or process.

14. A machine according to claim 1, further comprising:
at least one support for enveloping a relevant edge of the corrugated-board web, wherein the at least one support comprises a first leg, on which at least one microwave transmitter is arranged, the at least one support further comprising a second leg, on which at least one associated receiver unit is arranged, the at least one support further comprising an incline around an angle that substantially equals a flank angle of a corrugated web of the corrugated-board web, so that the microwaves are substantially transmitted through a flank of the corrugated web, the at least one support being independently set to a relevant flank angle of the corrugated web.

15. A microwave quality determining unit as a component of a machine according to claim 1, the microwave quality determining unit being configured such that at least one dielectric characteristic of a corrugated-board web is determinable.

16. A machine according to claim 1, wherein the at least one microwave quality determining means works with the microwaves, wherein a wavelength of the microwaves lies between 10 cm and 0.5 mm.

17. A machine according to claim 1, wherein the at least one microwave quality determining means works with the microwaves, wherein a frequency of the microwaves lies between 300 MHz and 300 GHz.

18. A machine according to claim 2, wherein the at least one microwave quality determining means works with the microwaves, wherein a frequency of the microwaves lies between 700 MHz and 100 GHz.

19. A machine according to claim 4, wherein the at least one microwave sensor comprises at least one microwave transmitter for generating a microwave field for interacting with the corrugated-board web.

20. A machine according to claim 19, wherein the at least one microwave sensor comprises at least one receiver unit for receiving the microwaves of the at least one microwave transmitter.

21. A machine according to claim 13, wherein areas or sections of the corrugated-board web determined by the at least one microwave quality determining means as deviating from a target condition of the corrugated-board web are marked.
